(19) United States
(12) Patent Application Publication
(10) Pub. No.: US 2026/0174351 A1
(43) Pub. Date: Jun. 25, 2026

(54) Body Region Dependent Evaluation of Medical Image Data

(71) Applicant: Siemens Healthineers AG, Forchheim (DE)

(72) Inventor: Mario Zeller, Erlangen (DE)

(73) Assignee: Siemens Healthineers AG, Forchheim (DE)

(21) Appl. No.: 19/430,657

(22) Filed: Dec. 23, 2025

(30) Foreign Application Priority Data
Dec. 23, 2024 (DE) 10 2024 212 288.2

(51) Int. Cl. A61B 5/055; A61B 5/00; G06T 5/60; G06T 5/70; G06V 10/70; G06V 10/762; G06V 10/774; G06V 10/82; G06V 40/10; G16H 50/20

(52) U.S. Cl. CPC A61B 5/055; A61B 5/4872; A61B 5/7267; A61B 5/7289; G06T 5/60; G06T 5/70; G06V 10/762; G06V 10/774; G06V 10/82; G06V 10/87; G06V 40/10; G16H 50/20; A61B 2576/00; G06T 2207/10088; G06T 2207/20004; G06T 2207/20081; G06T 2207/20084; G06T 2207/20092; G06V 2201/03

(57) ABSTRACT

A computer-implemented method for evaluating an image data set that is based on a medical imaging method, including: acquiring the image data set; acquiring a body region depicted in the image data set; processing the image data set using a processing algorithm, wherein at least one processing parameter of the processing algorithm is specified depending on the determined body region; and/or checking a trigger condition, whose fulfillment depends on whether, for at least one area of image data, which is reconstructed depending on the image data set, local geometric factors of the parallel magnetic resonance imaging used as the medical imaging method indicate a sufficiently strong local reduction in the signal-to-noise ratio, wherein an anatomical feature is assigned to the respective area depending on the body region and the image data set when the trigger condition is fulfilled.

### TECHNICAL FIELD

The disclosure relates to a computer-implemented method for evaluating an image data set that is based on a medical imaging method. In addition, the disclosure relates to a computer-implemented method for training a model trained using machine learning, a processing device, a magnetic resonance device, and a computer program.

### BACKGROUND

Image processing methods based on deep learning, for example, image reconstruction methods such as Deep Resolve Boost, usually give the user the opportunity to adjust the denoising level or other processing parameters, for example, a fat admixture with the Dixon technique. A choice is thereby typically offered between a few default settings, for example, the opportunity to choose between low, moderate, and strong denoising. The specific parameterization of the image reconstruction, or rather image processing in general, can also depend on the field strength of the main magnetic field used in the magnetic resonance imaging. So the same default setting at a field strength of 0.55 T can lead to considerably stronger denoising and/or noticeably lower addition of artificial noises than at a field strength of 3 T.

In the clinical environment, however, it has been determined that a low number of available default settings is often insufficient for adjusting the processing or denoising for all relevant imaging tasks to the wishes of the individual user, for example, of different radiologists. Specifying a higher number of default settings or using a higher number of adjustable parameters does lead to higher complexity in daily clinical routines, however, and is perceived as disruptive by many users.

The use of deep learning-based image processing methods or iterative reconstruction approaches in the field of magnetic resonance imaging can also lead to a misleading image impression under certain circumstances. For example, stronger local noise, as can arise during parallel magnetic resonance imaging in particular, can be heavily suppressed through deep learning-based or iterative reconstruction approaches so that an observer is given the impression of a much higher image quality, for example, a much lower acceleration factor in the imaging. However, this can lead to incorrect assessment of the image.

### SUMMARY

An object of the disclosure is thus to provide a user with better support for evaluating an image data set that is based on a medical imaging method.

The object is resolved according to the disclosure by a computer-implemented method for evaluating an image data set that is based on a medical imaging method, comprising the following steps:
acquiring the image data set,
acquiring a body region depicted by the image data set, and
processing the image data set by means of a processing algorithm, wherein at least one processing parameter of the processing algorithm is specified depending on the determined body region, and/or
checking a trigger condition, whose fulfillment depends on whether, for at least one area of image data, which is reconstructed depending on the image data set, local geometric factors of the parallel magnetic resonance imaging used as the medical imaging method indicate a sufficiently strong local reduction in the signal-to-noise ratio, wherein an anatomical feature is assigned to the respective area depending on the body region and the image data set when the trigger condition is fulfilled.

It was recognized that using a few possible default settings to parameterize the processing algorithm is problematic in particular, because one of the possible default settings is optimal in this instance depending on the depicted body region. By evaluating user behavior for a reconstruction resolution used by way of example with three possible settings for the denoising level, it was recognized in the context of the disclosure, however, that the strongest denoising is essentially always used for breast images, while minimal denoising is essentially always used for abdominal images. As typical users are thus already using maximum or minimal denoising, there is no possibility of increasing or reducing the denoising level further, in order to adjust it to the preference of a specific user.

As the parameterization of the processing algorithm to the depicted body region can be adjusted automatically in the disclosed method, however, such a manual setting of the denoising level by the user could theoretically be omitted altogether, which would simplify evaluation.

Particularly preferably, the automatic consideration of the body region when specifying the processing parameters is, however, combined with the user being able to adjust different denoising or processing levels. Thus, better image quality and better adjustability of the image properties to user wishes can be achieved. In particular, each of the possible default settings can be assigned different values for the processing parameters, for example, for weak, moderate, or strong denoising, depending on the depicted body regions. In the simplest case, a two-dimensional lookup table can be used, for example, which assigns a value to a pair of the default setting and depicted body region for each of the processing parameters. Other implementation possibilities are explained later.

By alternatively or additionally assigning areas, for which strong noise is predicted, to anatomical features, a note can be provided in particular to a user, for example, by means of a text message or voice output, which can explain in a manner easily understandable for medically trained users the areas in which there is a potential false image impression due to high noise.

By assigning the area heavily affected by noise to an anatomical feature and by naming this feature in the note, the user can in particular immediately see whether an impairment of image quality in the named area is relevant for a current evaluation task, for example, a diagnosis, or whether no or just a few relevant areas are affected for the current evaluation task. For example, an appropriate note might read: “Strong noise amplification in slice 7-9: medial, in the brain stem, moderate noise amplification in slice 12-14: left posterior, close to the occipital cortex.”

The depicted body region can be seen as an additional parameter in the context of determining the assigned anatomical area, whereby the robustness of the assignment can be increased. Particularly preferably, depending on the depicted body region, different partial algorithms can be selected, however, which are suitable for assigning the anatomical feature to the respective area for a specific body region. In this instance, the advantage is also achieved that the individual partial algorithms can be considerably less complex than a processing algorithm, which can carry out such an assignment for any body regions. This can reduce a necessary computing time and also simplifies the parameterization of the respective partial algorithm, which can be effected through machine learning, for example.

Locally differing noise levels can, for example, occur during magnetic resonance imaging if parallel imaging is used, in which measurement data from multiple measuring coils is combined to facilitate accelerated scanning of the k space. It is known for such imaging to determine a so-called g-factor map or geometric factor map, which describes the local g-factors or geometric factors, i.e., the respective local noise amplification based on the geometric arrangement of the receiving coils used for the parallel imaging. Possibilities for determining such a g-factor map can be found, for example, in articles by K. P. Pruessmann et al., “SENSE: Sensitivity Encoding for Fast MRI,” Magnetic Resonance in Medicine 42 (1999), pages 952 to 962, and F. A. Breuer et al., “General Formulation for Quantitative g-factor Calculation in GRAPPA Reconstruction,” Magnetic Resonance in Medicine 62 (2009), pages 739 to 746. The cited article by F A. Breuer et al. discloses the calculation of a g-factor map, from which weights used with a GRAPPA reconstruction can be determined in turn on the basis of a preparatory scan with low resolution.

Thus, in the context of evaluating the trigger condition, the preparatory scan can, for example, be used initially to determine the geometric factors, for example, as a g-factor map, which can, as will be explained later in more detail, be used to check whether or for which area or areas a strong noise is expected. As the imaging geometry of the preparatory scan should at least approximately correspond to the imaging geometry used for the final imaging, the preparatory scan can also be used as the image data set utilized by the processing algorithm to assign the anatomical feature to the respective area. Existing anatomical features in the preparatory scan in particular can be detected and localized for this purpose, whereby it can be determined within or in the proximity of which anatomical feature a respective area is located, for which strong noise is predicted. In order to improve the robustness of the detection and localization of anatomical features, the latest reconstructed image data itself can, however, also be processed as the image data set by the processing algorithms.

The body region depicted by the image data set can, for example, be acquired as a result of being provided with the image data set. For example, the depicted body region can be saved in the metadata assigned to the image data set or can come from a database that is also the origin of the image data set. However, it is also possible to acquire the body region as a result of it being manually entered or selected by the user. Particularly preferably, the depicted body region can be automatically determined and thus acquired by evaluating the image data set or another image data set, as will be explained later. The other image data set can depict the same patient with the same scan geometry as the image data set and come from a prescan, for example.

A model trained using machine learning can be utilized to determine the processing parameter, which uses the determined body region as part of its input data. In general, a model trained using machine learning mimics cognitive functions that humans associate with the thought processes of other humans. Training based on training data enables the trained model, in particular, to adapt to new conditions and to detect and extrapolate patterns. Another term for a “model trained using machine learning” is a “trained function.”

A trained model is particularly suitable for also learning and taking into account complex relationships between different input data. Using such a trained model is thus particularly expedient if other input variables need to be taken into account in addition to the acquired body region, in order to determine the at least one processing parameter. In principle, it would be possible in this instance to use a multi-dimensional lookup table, for example, to determine the processing parameter. However, this would potentially be very extensive when taking into account a lot of input variables and/or with a high resolution of at least some of the input variables, whereby generating such a lookup table would require substantial testing and analysis. There is also a discussion below of the relevant input variables that it can be expedient to take into account when determining the processing parameter, and that therefore can be used as part of the input data for the trained model.

In general, the parameters of a machine-learning model can be adjusted through training, in order to provide the trained model. The training can, in particular, be upstream of the method according to the disclosure and thus not be part of the method according to the disclosure. Alternatively, it would also be possible, however, for training to be carried out as additional upstream method steps within the method according to the disclosure.

In particular, supervised training, semi-supervised training, unsupervised training, reinforcement learning, and/or active learning can be used. Furthermore, representation learning, also known as “feature learning,” can be used. In particular, the parameters of the machine-learning models can be adapted iteratively through multiple training steps. In particular, a specific cost function can be minimized in the context of the training. In particular, the backpropagation algorithm can be used when training a neural network, for example.

A machine-learning model can comprise, in particular, a neural network, a support vector machine, a decision tree, and/or a Bayesian network, and/or a transformer, and/or the machine-learning model can be based on k-means clustering, Q-learning, genetic algorithms, and/or association rules. In particular, a neural network can be a deep neural network, a convolutional neural network (CNN), or a convolutional deep neural network. Furthermore, a neural network can be an adversarial network, a deep adversarial network, and/or a generative adversarial network.

The respective processing parameter can be provided directly by the trained model, 1 or this can, for example, provide an offset or a scaling factor for a respective specified base value of the respective processing parameter. Insofar as a user should be able to choose a default setting for the denoising or processing level, as explained above, the selected default setting can be processed in this instance as part of the input data.

Alternatively or additionally, the trained model used for the determination can be chosen from multiple different trained models, depending on the selected default setting. In this instance, a first trained model can, for example, provide the respective processing parameter for a high denoising or processing level, a second trained model the respective processing parameter for a moderate denoising or processing level, and a third trained model the respective processing parameter for a low denoising or processing level.

Using separate trained models means, for example, that training data can be generated for supervised learning, by different example data sets each being processed with different parameterizations of the processing algorithm and the denoising or processing level then being assessed, for example, by human experts or also automatically using the processing result, for example, of the image data set reconstructed in accordance with the respective processing parameters.

A training data set can then be provided for each processing result whose denoising or processing level was assessed as high, which can be used in a known manner for supervised training of the first trained model. On the one hand, the respective training data set can comprise relevant input variables, i.e., at least the depicted body region in the example data set underlying the processing result and, optionally, other input variables that are explained later and, on the other hand, the management parameters used to determine this processing result, or offsets, or scaling factors for notable instances of these processing parameters. As part of the training, error feedback, for example, the gradient descent method, can be used to minimize a deviation of the processing parameters, or offsets, or scaling factors determined by the first trained model from the variables specified in the respective training data set.

The second and third trained model can be trained in the same way, wherein processing results whose denoising or processing level is assessed as moderate or low are gathered in each case to provide the training data sets. The use of three different denoising or processing levels is purely an example, and it would also be possible to take into account just two or more than three different denoising or processing levels.

As the variability is relatively low, even when taking into account the other possible input data explained later, classic approaches of machine learning can be used, for example, a random forest regressor or a simple neural network, for example, a fully connected neural network with one or two hidden layers with 16-32 neurons and ReLU activation networks. For example, one to three output neurons can be used to provide the processing parameters or offsets for these.

The processing algorithm that processes the image data set can comprise multiple partial algorithms, of which one is selected to process the image data set, wherein an identifier assigned to the selected partial algorithm forms part of the input data of the trained model.

The at least one processing parameter determined by means of the trained model is preferably abstract enough to parameterize different partial algorithms and even different processing types implemented by these. For example, when using a reconstruction algorithm as the respective partial algorithm, a noise suppression number, for example, between 0 and 1, and optionally also a proportion for an addition of artificial noise can be specified as processing parameters for the reconstructed image data. Even when using another of the partial algorithms, the same trained model can still be used to specify the parameters, wherein fine-tuning, for example, of the provided processing parameters to the selected partial algorithm is possible, however, by taking into account the identifier as input data.

A user can select a partial algorithm manually in the simplest case. However, the choice is preferably effected automatically, for example, in the case of magnetic resonance imaging depending on a field strength of the main magnetic field and/or a sequence type used.

In particular, the partial algorithms or at least sections of these can be or comprise a respective other model trained using machine learning. In this instance, the identifier can, for example, describe the type of trained model that has been used, i.e., what kind of neural network is being used as the partial algorithm, for example.

The image data set can be determined through magnetic resonance imaging. Due to the varied imaging and parameterization possibilities of magnetic resonance imaging and particularly due to the fact that significantly different parameterizations of the processing algorithm may be necessary, depending on the depicted body region and form of the imaging process for a processing result, which usefully will typically be assessed as moderately denoised or processed, the method according to the disclosure is particularly expedient for image data sets in magnetic resonance imaging. However, it can also be used in principle for image data sets for other medical imaging modalities, for example, computed tomography, ultrasound imaging, or molecular imaging.

The processing parameters, or at least one of the processing parameters, can additionally be determined depending on at least one of the following input variables:
a sequence type of the measuring sequence used for the magnetic resonance imaging,
a repetition time of the measuring sequence,
an echo time of the measuring sequence,
a method for suppressing a fat signal used in the context of the measuring sequence,
an acceleration factor of the measuring sequence,
a field strength of the main magnetic field, and/or
a type of reference scan used in the context of parallel imaging.

The stated input variables have proven particularly relevant in the selection of processing parameters in the context of the disclosure. In particular, the at least one input variable can be part of the input data for the model trained using machine learning explained above, which is utilized to determine the processing parameter.

Sequence types may include spin-echo sequences, inversion recovery sequences, gradient echo sequences, various functional imaging sequences, and/or subtypes of the listed sequence types.

Different methods for suppressing a fat signal may include fat suppression using the chemical shift between water and fat, frequency-selective fat suppression, T1 time-dependent fat suppression, and/or an inversion technique for suppressing the fat signal.

An acceleration factor can indicate, for example, a ratio between the number of k-space points, which are based on a reconstructed image with the given resolution, and the number of actually scanned k-space points.

Reference scans are used in the context of parallel imaging in particular to determine weights for the measuring signals of the different measuring coils at different points. A differentiation can be made here, for example, between a low-resolution acquisition of the entire image area and the acquisition of an individual high-resolution central line.

The depicted body region can be determined by evaluating the image data set or another image data set. In principle, it would be possible, for example, to derive the depicted body region from user information, for example, in the context of patient registration. However, this information may be incorrect or ambiguous, for example, if multiple body regions are examined in succession or if phantoms are scanned.

In one preferred aspect variant, the body region is therefore determined automatically by evaluating or the other image data set, for example, by means of a model trained using machine learning, for example, by means of a simple classifier network, or through typical classification approaches, for example, using a scale-invariant feature transformation. In addition to the image data set ultimately used for the imaging, another image data set acquired in the context of a prescan or an adjustment measurement can also be used, as detection of the body region is already possible at very low resolution or with heavy undersampling of the k space.

A level of noise suppression performed in the context of the processing, and/or an addition of an artificially produced noise component, and/or a degree of fat admixture can be specified with a fat suppression as the processing parameter or as one of the processing parameters.

Adding an artificially produced noise component can be expedient with certain denoising approaches or reconstruction approaches in order to produce a natural image impression and thus make it easier for a user to detect relevant structures.

In magnetic resonance imaging in particular, amounts of fat are suppressed in many imaging tasks through suitable measuring methods and suitable measurement data preparation. However, it can be expedient to retain a certain amount of fat in the image for certain body regions or depending on other input variables.

The processing algorithm can determine output data, wherein the output data or an image representation based on the output data can be output to a user via an output device, after which a user input relating to an assessment of the output data or the image representation can be acquired from the user, after which another image data set can be received and processed by the processing algorithm, wherein the processing parameter of the processing algorithm can be specified in the process depending on the user input.

A user-specific fine-tuning of the processing can be achieved using the described method. The user can select, for example, by pressing a corresponding button or section of the touchscreen or by selecting a corresponding menu item, as user input, whether they found the achieved denoising or processing level too weak, just right, or too strong. Depending on the user input, for example, if the processing is too weak or too strong, a fixed offset can then be added to the processing parameter determined as explained above, or subtracted from it, in order to process the other image data set. This offset can be stored in a database, particularly on a user-specific basis, but alternatively also on a device-specific basis, for example, and can always be added or subtracted for this parameter combination. Alternatively or additionally, it would also be possible, for example, to gather corresponding user feedback, i.e., corresponding user input, about the processing of multiple different image data sets and to use the gathered data, for example, for further training of the model trained using machine learning that is utilized to determine the processing parameter.

A voice message or text message naming the anatomical feature can also be output to the or a user when the trigger condition is fulfilled. This was already discussed at the beginning.

A cluster analysis can be performed to evaluate the trigger condition, in order to detect clusters of a geometric factor map describing the local geometric factors, within which all geometric factors achieve or exceed a specified threshold, wherein the clusters whose dimensions fulfill a specified selection condition are selected as the respective area to which an anatomical feature is assigned.

Thus, after determining the g-factor map, which can, for example, be based on a prescan as explained above, regions affected by a noticeable noise amplification can be identified, an anatomical feature can be assigned to these regions, and the anatomical feature can, for example, be output as part of a note, as explained above.

Once the geometric factor map is available, it can optionally be checked first to determine whether the entire image is impaired by a low signal-to-noise ratio. Voxels or pixels in the geometric factor map that reach or exceed a set threshold, for example, 2 or 3, can be counted for this purpose. If a set percentage of the voxels or pixels, for example, 50% of the voxels or pixels, exceeds this value, a general message can be output that the noise amplification is very high, and the procedure can end.

If this threshold is not reached, or if the optional check explained above is not performed, the voxels or pixels of the geometric factor map can be masked so that only the voxels or pixels that exceed the threshold are selected. It can then be determined which voxels or pixels belong to clusters. Known methods can be used for this purpose, such as k-means clustering or DBSCAN (density-based spatial clustering of applications with noise).

The selection condition can, for example, be fulfilled for clusters that have a specified minimum size, for example, a size of at least 20 pixels or voxels, and/or that extend across multiple slices in three-dimensional image data sets.

Optionally, an average geometric factor of the identified clusters can be determined so that the clusters can be sorted by their average geometric factors. This can be used, for example, in a note to users to indicate only the cluster or clusters with the highest average geometric factor.

A detection algorithm, which is embodied by a model trained using machine learning, can detect anatomical features in the image data set or in image data that is reconstructed depending on the image data set, wherein the selection of the detection algorithm depends on the depicted body region, and/or wherein the depicted body region is processed as part of the input data of the detection algorithm, wherein the area is assigned to one of the detected anatomical features depending on the relative position of the respective area in relation to the detected anatomical features.

By using separate detection algorithms or additionally taking into account the depicted body region, the robustness of the detection of anatomical features can be improved considerably. Additionally, relatively simple models that can be trained with little effort can be used, particularly when using separate detection algorithms for different body regions. Automatic detection of anatomical features is known and does not therefore need to be explained in detail.

In cases in which no clear assignment is possible, purely geometric information can, for example, be produced in the alert message explained above, such as “Slice 12-14: left posterior.” Typically, however, the information can include the specific anatomical region, such as “Slice 12-14: left posterior, close to the occipital cortex.”

The disclosure also relates to a method for training a model trained using machine learning, which is utilized in the disclosed computer-implemented method for evaluating an image data set to determine the at least one processing parameter of the processing algorithm and comprises the following steps:
acquiring multiple training data sets, each comprising input data and target output data for the trained model, wherein the target output data specifies a respective target value for the respective processing parameter to be determined,
training the trained model on the basis of the input data and the target output data,
providing the trained model.

The trained model used to provide the at least one processing parameter can thus be trained in a known manner using supervised learning when suitable training data sets are available. In particular, iterative training can be used, wherein a deviation of the actual output data from the target output data can be reduced in the customary manner through error feedback, for example, using the gradient descent method.

As already explained in relation to the computer-implemented method for evaluating an image data set, the training can, for example, be applied to a random forest regressor or a simple neural network, for example, a fully connected neural network with one or two hidden layers with 16-32 neurons and ReLU activation networks. The provision of suitable training data sets has likewise already been discussed.

The disclosure also relates to a processing device, which is programmed to carry out the disclosed computer-implemented method for evaluating an image data set and/or to carry out the disclosed computer-implemented method for training a model trained using machine learning.

The disclosure also relates to a magnetic resonance device, which comprises the processing device according to the disclosure. As already explained above, the disclosed method is particularly expedient for magnetic resonance measurements, and integration in the magnetic resonance device can minimize the effort required to use this method. Alternatively, however, the processing device can also be embodied separately from the magnetic resonance device, for example, as a workplace computer, server, or cloud solution.

The disclosure also relates to a computer program with instructions, which is programmed to carry out the disclosed computer-implemented method when executed on a data processing device.

The disclosure also relates to a data carrier, which comprises the computer program according to the disclosure.

Independent of the grammatical term usage, individuals with male, female, or other gender identities are included within the term.

### BRIEF DESCRIPTION OF THE DRAWINGS

Further advantages and details of the disclosure are specified in the following exemplary aspects and the associated drawings. The figures show the following schematic representations:

FIG. 1 illustrates an exemplary aspect of a magnetic resonance device, which comprises an exemplary aspect of a processing device,

FIGS. 2 and 3 illustrate flow charts of exemplary aspects of the disclosed method for evaluating an image data set,

FIG. 4 illustrates example image data processed in the method according to FIG. 3,

FIG. 5 illustrates a flow chart of an exemplary aspect of the disclosed computer-implemented method for training a model trained using machine learning, and

FIG. 6 illustrates a possible structure of the model trained using machine learning, utilized to determine the processing parameters in the method according to FIG. 2.

### DETAILED DESCRIPTION

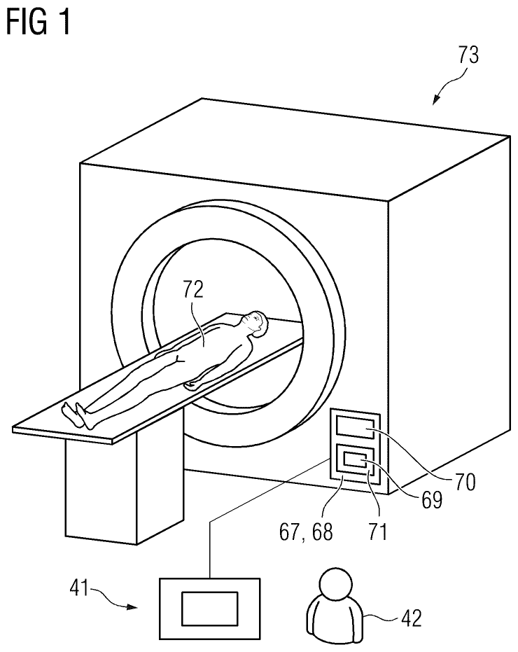

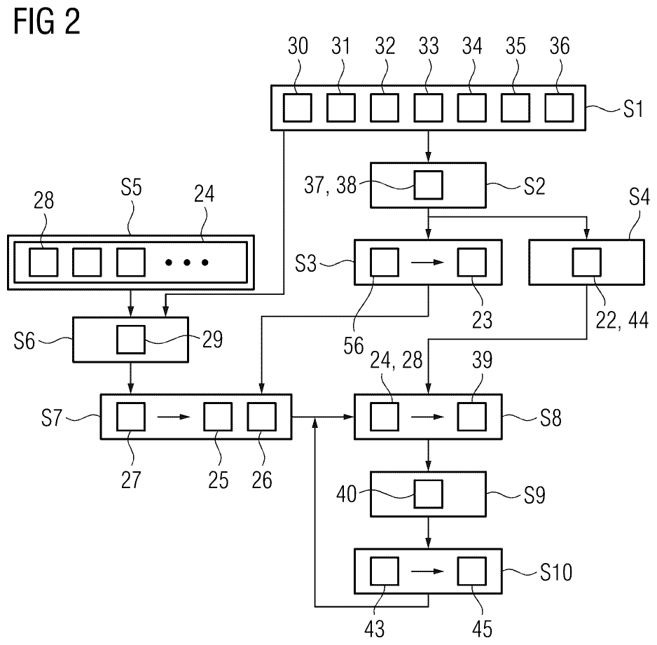

FIG. 1 shows a magnetic resonance device 73, which can be used to determine an image data set 22, which depicts the patient 72. In the example, a processing device 67, which is integrated into the magnetic resonance device 73, evaluates the image data set 22; this evaluation is described in more detail below. The processing device 67 is programmed in the example to carry out the method for evaluating the image data set 22, which is explained below with reference to FIGS. 2 and 3. Alternatively, however, this method could also be implemented by a processing device 67 embodied separately from the magnetic resonance device 73, for example, by a workplace computer, a server, or a cloud solution.

In the example, the processing device 67 is implemented by a programmable data processing device 68 with a processor 70 and a memory 71, wherein a computer program 69 is stored in a memory 71, whose instructions implement the computer-implemented method explained below with reference to FIGS. 2 and 3 when executed.

Both of the methods explained below, with reference to FIGS. 2 and 3, involve a consideration of the example of a body region 23 depicted by the image data set 22, in order to optimize image quality or to advise a user of potential image quality defects. The selection of processing parameters or the advising is explained below using the example of parallel imaging by means of multiple receiving coils of the magnetic resonance device. As already explained in the general section, the explained selection of processing parameters depending on body region, in particular, is, however, also extremely useful for other examination modalities.

It can be advantageous to carry out the two methods discussed with reference to FIGS. 2 and 3 together so that, for example, an image representation 40 can be reconstructed for the user 42 by means of the method shown in FIG. 2, while in parallel, by means of the method shown in FIG. 3, the user 42 can be informed in the event of an expected strong local reduction in the signal-to-noise ratio that, or rather in the area of which anatomical feature, the image impression is potentially being distorted. The methods are discussed separately below to simplify the understanding of the relevant flows. To improve understanding of the overall context, the imaging itself is shown as part of the method. However, it is of course also possible to carry out the imaging outside the method and to access image data sets stored in a database, for example, as part of the evaluation.

The general objective of the method shown in FIG. 2 is that, when an image data set 22 is processed by a processing algorithm 24, its processing parameters 25, 26, particularly at least one denoising or processing level, are specified depending on a body region 23 depicted by the image data set. In the example, the processing serves to determine an image representation 40, which can be output to the user 42 by means of the output device 41.

In step S1, different parameters are first specified for the imaging in the example, namely a sequence type 30 of the measuring sequence used for the magnetic resonance imaging, a repetition time 31 of the measuring sequence, an echo time 32 of the measuring sequence, a method 33 for suppressing a fat signal used in the context of the measuring sequence, an acceleration factor 34 of the measuring sequence, a field strength 35 of the main magnetic field, and a type 36 of a reference scan 37 used in the context of parallel imaging. These parameters have already been explained in more detail in the general sections. In variations of the method, it would also be possible to consider only some of these parameters or additional imaging-relevant parameters.

A reference scan 37 is then first acquired in step S2. Depending on the type 36 of reference scan 37 that is used, such a reference scan 37 may, for example, have a lower resolution than the image data set 22 and/or the image data acquisitions may only be one-dimensional or two-dimensional.

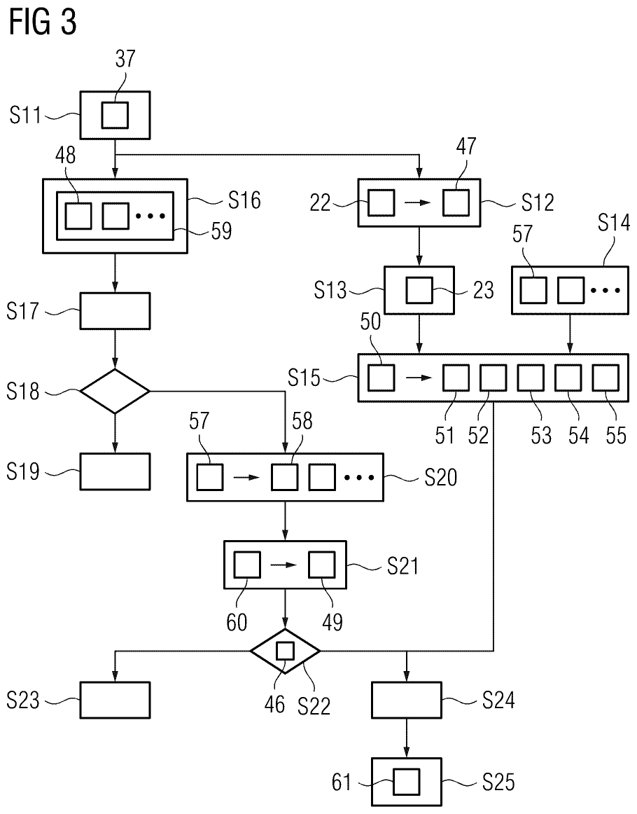

In the example, the reference scan is then used in step S3 as another image data set 38, on the basis of which the depicted body region 23 is automatically determined. As shown in FIG. 4, an image of the head of the patient 72 is used as an example of the body region 23. In alternative aspects, it would be possible to determine the body region 23 by evaluating the image data set 22, for example, using a preliminary reconstruction.

As there are typically only a few body regions to differentiate between, for example, between the head, abdomen, chest area, and limbs, a relatively simple algorithm 56 can be used in step S3 to determine the body region 23, for example, a simple classifier network trained using machine learning.

In step S4, the image data set 22 to be processed is acquired, for example, by means of known parallel magnetic resonance imaging.

Steps S5 to S7 involve selecting a processing of the image data set 22 and parameterizing it appropriately. In the example, it is assumed that the processing algorithm comprises multiple partial algorithms 28, which are provided in step S5. In step S6, depending on the field strength 35 of the main magnetic field, one of these partial algorithms 28 or rather the assigned identifier 29 is then selected, which will process the image data set in step S8. A specified lookup table, for example, can be used for the selection process.

In step S7, processing parameters 25, 26 of the processing algorithm 24, or rather the partial algorithms 28, are then specified depending on the determined body region 23. This is effected in the example by a model 27 trained using machine learning. In addition to the body region 23, the input data processed by the trained model 27 in the example also comprises the identifier 29 of the partial algorithm 28 that has been used and the variables specified in step S1.

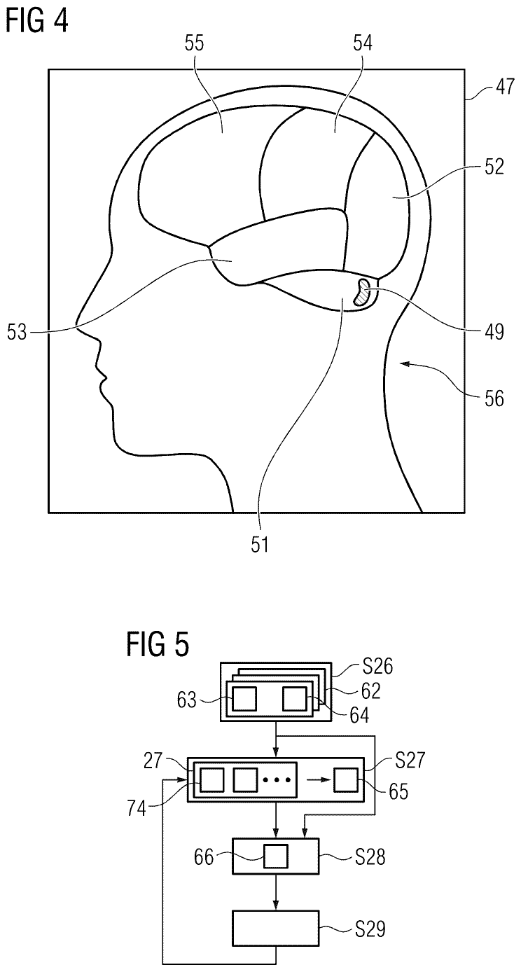

As already discussed in the general section, a model 27 trained using machine learning can also be utilized to take into account complex relationships between the different sections of the input data, in order to determine suitable processing parameters 25, 26. A possible training approach for the trained model 27 will be explained later with reference to FIG. 5.

In the example, the processing parameter 25 specifies the level of denoising in the context of reconstructing the output data 39. For example, the processing parameter 25 can specify a frequency limit, a filtering, a selection of a used filter kernel, denoising factors with iterative reconstruction methods, or similar. The processing parameter 26 in the example specifies how much artificial noise has been added to the reconstruction result. Such a noise addition can be expedient, in order to adjust the output data 39, or rather an image representation 40 determined therefrom, to a desired image impression for the user 42.

As already explained in the general section of the description, it is possible for the user 42 to specify a desired denoising level, for example. This could be incorporated as another parameter in step S1, for example, and be fed into the trained model 27 as an additional part of the input data. As likewise already discussed in the general section, it can be advantageous, however, instead to choose one of multiple trained models 27 that have been trained for the desired denoising level in each case, depending on the user's desired denoising level.

In step S8, the image data set 22 is then processed by the processing or partial algorithm 24, 28 parametrized according to the processing parameters 25, 26, in order to provide the output data 39. For example, the output data can describe a reconstructed three-dimensional or two-dimensional image.

In step S9, an image representation 40, for example, a two-dimensional sectional image, is then determined on the basis of the output data 39, which is output to the user 42 via the output device 41.

In step S10, a user input 43 of the user 42 is acquired, which relates to an assessment of the image representation 40. In particular, users can report whether they believe the affected denoising is too strong or too weak, for example, by operating a corresponding area of the output device embodied as a touchscreen or by using separate buttons or other control elements. When another image data set 44 is subsequently processed, at least one of the processing parameters 25, 26 can then be specified, depending in addition on the user input 43.

For example, depending on the user input 43, a fixed offset 45 can be added to one of the processing parameters 25, 26, in order to take into account that the user apparently desires stronger or weaker denoising. As already explained in the general section of the description, it would also be additionally or alternatively possible to use the user input 43 in further training of the trained model 27, so that, for example, a model trained generally at first can be adjusted to certain users or user groups through further training.

The method explained above, or also other processing or denoising approaches that perform iterative processing and/or are based on machine learning, for example, can also be used to give the user 42 an impression of a high-quality image in some circumstances if the actual image data is strongly inverted. The objective of the method shown in FIG. 3 is thus to output in this instance not only a general note highlighting a noise, a text message 61 in the example, but rather to inform the user in this note of the area in which an anatomical feature or within which anatomical features where corresponding image impairment is to be expected. This enables the user 42, for example, to assess immediately whether the image impairment is relevant for a current evaluation task, for example, for making a diagnosis.

The example shown begins in step S11 with the acquisition of a reference scan 37. The acquisition of a reference scan 37 has already been explained in relation to step S2 in the method shown in FIG. 2.

In step S12, the image data set 22 is then determined, as already explained above in step S4, and the image data 47 is reconstructed on the basis of the image data set 22. As parallel imaging is performed in the example, as explained above, local weighting factors for the signal contributions of the individual receiving coils are taken into account in the context of the reconstruction, which can be averaged on the basis of the reference scan 37.

In step S13, the depicted body region 23 is then determined on the basis of the image data 47, for example, by means of a simple classifier network trained using machine learning as explained in step S3. As represented schematically in FIG. 4, the head of the patient 72 is depicted in the example.

Steps S14 and S15 serve to detect and localize anatomical features 51-55, in the example, different regions of the brain. A convolutional neural network (CNN) specific to a body region is used for this in the example.

Using different detection algorithms 57 for different body regions 23 means that a relatively simple and thus easy to train convolutional neural network can be used as the detection algorithm 57 in each instance. Therefore, different potential detection algorithms 57 are first provided in step S14, which are each trained to detect anatomical features in a specific body region 23. The respective detection algorithm can be trained in particular by means of supervised learning. Suitable training data sets can be provided, for example, by means of medical specialists segmenting and classifying the respective anatomical regions depicted in image data used for training. The training can then proceed in the customary manner by means of error feedback, for example, using the gradient descent method.

In step S15, a selection is made from the potential detection algorithms 57 of the detection algorithm 50 that is assigned to the body region 23 detected in step S13, in order to localize the anatomical features 51-55 depicted in the image data 47.

In parallel to the detection of anatomical features 51-55, a geometric factor map 59 is determined in the example in step S16, which describes local geometric factors 48, which describe the expected local reduction of the signal-to-noise ratio in the image data 47 due to the parallel imaging. It is assumed in the example that the geometric factor map 59 is determined from the weighting factors used in the context of the reconstruction in step S12, which are in turn averaged from the reference scan 37. A detailed representation of such a method can be found, for example, in the work of F. A. Breuer et al. cited at the beginning. Alternatively, however, any other known or new approach for determining the geometric factor map 59 can be used.

In step S17, all points or voxels in the geometric factor map 59 are then selected, whose value reaches or exceeds a specified threshold, for example, 2 or 3, i.e., an expected reduction in the signal-to-noise ratio by a factor of 2 or 3.

In step S18, a check is then performed to determine whether the number of these points or voxels exceeds a specified threshold, i.e., 50% of the total number of pixels or voxels, for example. If this is the case, a general note, for example, a text message, is output on the output device 41 in step S10, stating that a very high increase in noise is present.

If, by contrast, this is not the case, a cluster analysis 57 is carried out in step S20, for example, using k-means clustering or DBSCAN, in order to detect clusters 58 in the geometric factor map 59, within which all geometric factors 48 reach or exceed the specified threshold. The detected clusters 58 are then checked to determine whether their dimensions fulfill a specified selection condition 60. In the example, cluster 58 then fulfills the trigger condition if it comprises a specified number of pixels or voxels, and additionally, in the case of three-dimensional imaging, if the respective cluster 58 extends across multiple slices.

If none of the detected clusters 58 fulfill the selection condition 60, the trigger condition 46 in step S22 is not fulfilled, and the reconstructed image data 47 or an image representation determined from it can then, for example, be output via the output device 41 with no further note in step S23.

If at least one of the clusters 58 fulfills the selection condition 60, the trigger condition 46 in step S22 is fulfilled, and at least one of the anatomical features 51-55 detected in step S15 is assigned to this cluster 58 or the area 49 of the image data 47 that corresponds to this cluster 58 in step S24. In the example, as can be seen in FIG. 4, the area 49 lies in the area of the anatomical feature 51, i.e., in the brain stem. Optionally, for further specificity, it can be noted that the area 49 lies close to the anatomical feature 52.

In step S25 in the example, a text message 61 is then output to the means of display, which comprises the anatomical feature 51 assigned to the area 49, for example, in addition to the image data 47 or an image representation generated from it. As already explained in the general section, it is possible to specify a slice or another geometrical localization as well. A message can thus be output, for example: “Strong noise amplification in slice 7-9: medial, in the brain stem.”

A possible approach to training the trained model 27 used in step S7 in FIG. 2 is explained below with reference to FIG. 5.

In step S26, multiple training data sets 62 are provided, which each comprises the input data 63 and target output data 64 for the trained model 27. The target output data 64 specifies a respective target value in the example for each of the processing parameters 25, 26 to be determined. The input data corresponds to the input data already explained with reference to FIG. 2. Possibilities for providing suitable training data sets 62 have already been explained in the general section of the description and are therefore not repeated.

In steps S27 to S29, the trained model 27 is then trained by means of known supervised training. The trained model 27 is applied in its initial state, in which its parameters 74 can have random values, for example, or in the current training state in each instance in later iterations, to the input data 63 of each training data set of a current batch of training data sets.

A cost function 66 evaluated in step S28 then corresponds to a measure for the deviations between the actual output data 65 determined for the respective training data set and the respective target output data 64. By means of the error feedback in step S29, for example, using the gradient descent method, the cost function 66 is minimized iteratively by varying the parameters 74.

When a convergence criterion is fulfilled, for example, after a specified number of iterations or if there is very little variation in the cost function 26 between iterations, the training is complete and the trained model 27 can then be provided for later use.

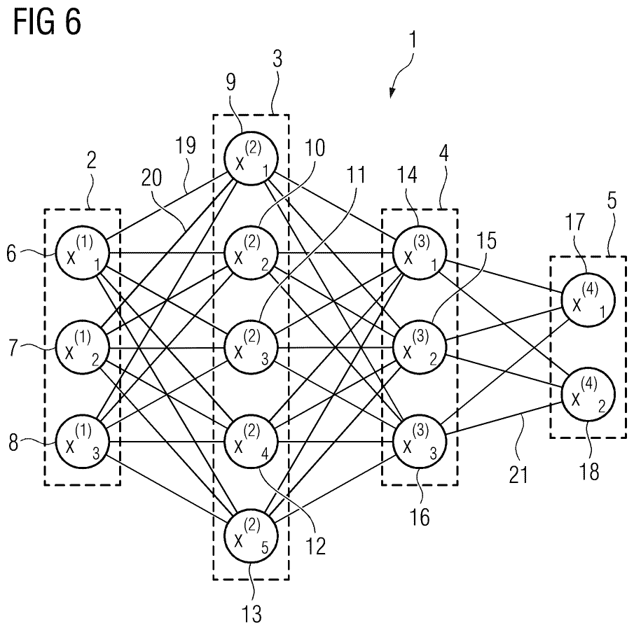

The trained model 27 that is used in step S7 in the example shown in FIG. 2 can, for example, be implemented by a neural network. To simplify matters, the properties of such a neural network are explained briefly below using a very simple example with reference to FIG. 6. As explained above, 16 to 32 artificial neurons in particular can be used in real applications, particularly to enable a greater amount of input data to be taken into account. Accepted terms for the artificial neural network 1 include “neural network,” “artificial neural net,” or “neural net.”

The artificial neural network 1 comprises nodes 6 to 18 and edges 19 to 21, wherein each edge 19 to 21 is a directed connection from a first node 6 to 18 to a second node 6 to 18. Generally, the first nodes 6 to 18 and the second nodes 6 to 18 are different nodes 6 to 18, but it is also conceivable for the first nodes 6 to 18 and the second nodes 6 to 18 to be identical. For example, in FIG. 1, the edge 19 is a directed connection from the node 6 to the node 9, and the edge 21 is a directed connection from the node 16 to the node 18. An edge 19 to 21 from a first node 6 to 18 to a second node 6 to 18 is known as the ingoing edge for the second node 6 to 18 and as the outgoing edge for the first node 6 to 18.

In this exemplary aspect, the nodes 6 to 18 of the artificial neural network 1 can be arranged in layers 2 to 5, wherein the layers can have an intrinsic order, which is established by the edges 19 to 21 between the nodes 6 to 18. In particular, edges 19 to 21 can only be provided between neighboring layers of nodes 6 to 18. In the illustrated exemplary aspect, there is an input layer 2 that only has nodes 6, 7, 8, each without an ingoing edge. The output layer 5 only comprises the nodes 17, 18, each without outgoing edges, wherein further hidden layers 3 and 4 lie between the input layer 2 and the output layer 5. The number of hidden layers 3, 4 can generally be selected freely. The number of nodes 6, 7, 8 of the input layer 2 usually corresponds to the number of input values in the neural network 1, and the number of nodes 17, 18 in the output layer 5 usually corresponds to the number of output values of the neural network 1.

In particular, a (real) number can be assigned to the nodes 6 to 18 of the neural network 1. x⁽ⁿ⁾; thereby denotes the value of the ith node 6 to 18 of the nth layer 2 to 5. The values of the nodes 6, 7, 8 of the input layer 2 are equivalent to the input values of the neural network 1, while the values of the nodes 17, 18 of the output layer 5 are equivalent to the output values of the neural network 1. Moreover, a weight in the form of a real number can be assigned to each edge 19, 20, 21. In particular, the weight is a real number in the range [−1, 1] or in the range [0, 1,]w^{(m,n)}_{i,j} thereby denotes the weight of the edge between the ith nodes 6 to 18 of the mth layer 2 to 5 and the jth nodes 6 to 18 of the nth layer 2 to 5. Furthermore, the abbreviation

$${w}_{i , j}^{( n )}$$

is defined for the weight

$${w}_{i , j}^{( n , n + 1 )} .$$

In order to calculate output values of the neural network 1, the input values are propagated by the neural network 1. In particular, the values of the nodes 6 to 18 of the (n+1)th layer 2 to 5 can be calculated on the basis of the values of the nodes 6 to 18 of the nth layer 2 to 5.

$${x}_{j}^{( n + 1 )} = f ( {\sum }_{i} {x}_{i}^{( n )} \cdot {w}_{i , j}^{( n )} ) .$$

Thereby, f is a transfer function that can also be called an activation function. Known transfer functions are step functions, Sigmoid functions (for example, the logistic function, generalized logistic function, hyperbolic tangent, arc tangent, error function, smoothstep function), or rectifier functions. The transfer function is predominantly used for normalization purposes.

In particular, the values are propagated step-by-step by the neural network 1, wherein values of the input layer 2 come from the input data of the neural network 1. Values of the first hidden layer 3 can be calculated on the basis of the values of the input layer 2 of the neural network 1, values of the second hidden layer 4 can be calculated on the basis of the values in the first hidden layer 3, etc.

The neural network 1 must be trained using training data, in order to be able to define the values

$${w}_{i , j}^{( n )}$$

for the edges 19 to 21. In particular, training data comprises training input data and training output data, which is referred to below as tᵢ. The neural network 1 is applied to the training input data for one training step, in order to determine calculated output data. In particular, the training output data and the calculated output data comprise a number of values, wherein the number is determined as the number of nodes 17, 18 of the output layer 5.

In particular, a comparison between the calculated output data and the training output data is used, in order to adjust the weights within the neural network 1 recursively (backpropagation algorithm). In particular, the weights can be altered according to

$${{w}^{′}}_{i , j}^{( n )} = {w}_{i , j}^{( n )} - γ \cdot {δ}_{j}^{( n )} \cdot {x}_{i}^{( n )}$$

wherein γ is a learning rate and the numbers

$${δ}_{j}^{( n )}$$

can be calculated recursively as

$${δ}_{j}^{( n )} = ( {\sum }_{k} {δ}_{k}^{( n + 1 )} \cdot {w}_{j , k}^{( n + 1 )} ) \cdot {f}^{′} ( {\sum }_{i} {x}_{i}^{( n )} \cdot {w}_{i , j}^{( n )} )$$

on the basis of

$${δ}_{j}^{( n + 1 )}$$

it the (n+1)th layer is not the output layer 5, and

$${δ}_{j}^{( n )} = ( {x}_{k}^{( n + 1 )} - {t}_{j}^{( n + 1 )} ) \cdot {f}^{′} ( {\sum }_{i} {x}_{i}^{( n )} \cdot {w}_{i , j}^{( n )} )$$

if the (n+1)th layer is the output layer 5, wherein “ƒ′” is the first derivation of the activation function and

$${y}_{j}^{( n + 1 )}$$

the comparative training value for the jth nodes 17, 18 of the output layer 5.

1. A computer-implemented method for evaluating an image data set that is based on a medical imaging method, comprising:
acquiring the image data set;
acquiring a body region depicted in the image data set; and
processing the image data set using a processing algorithm, wherein at least one processing parameter of the processing algorithm is specified depending on the acquired body region; and/or
checking a trigger condition, whose fulfillment depends on whether, for at least one area of image data, which is reconstructed depending on the image data set, local geometric factors of a parallel magnetic resonance imaging used as the medical imaging method indicate a sufficiently strong local reduction in signal-to-noise ratio,
wherein, when the trigger condition is fulfilled, an anatomical feature is assigned to the respective area depending on the acquired body region and the image data set.

2. The computer-implemented method as claimed in claim 1, wherein a model trained using machine learning is utilized to determine the processing parameter of the processing algorithm, wherein the acquired body region forms part of input data of the model.

3. The computer-implemented method as claimed in claim 2, wherein the processing algorithm that processes the image data set comprises a plurality of partial algorithms, one of which is selected to process the image data set, wherein an identifier assigned to the selected partial algorithm forms part of the input data of the model.

4. The computer-implemented method as claimed in claim 1, wherein the image data set is determined by magnetic resonance imaging.

5. The computer-implemented method as claimed in claim 4, wherein the processing parameter or at least one of the processing parameters is additionally determined based on:
a sequence type of a measuring sequence used for the magnetic resonance imaging;
a repetition time of the measuring sequence;
an echo time of the measuring sequence;
a method for suppressing a fat signal used in context of the measuring sequence;
an acceleration factor of the measuring sequence;
a field strength of the main magnetic field; and/or
a type of a reference scan used in context of parallel imaging.

6. The computer-implemented method as claimed in claim 1, wherein the acquired body region is determined by evaluating the image data set or another image data set.

7. The computer-implemented method as claimed in claim 1, wherein a level of noise suppression performed in context of the processing, an addition of an artificially produced noise component, and/or a degree of fat admixture is specified with a fat suppression as the processing parameter or as one of the processing parameters.

8. The computer-implemented method as claimed in claim 1, wherein the processing algorithm determines output data, wherein the output data or an image representation based on the output data is output to a user via an output device, and wherein a user input relating to an assessment of the output data or the image representation is acquired from the user, and wherein another image data set is subsequently received and processed by the processing algorithm, such that the processing parameter of the processing algorithm is specified in the process depending on the user input.

9. The computer-implemented method as claimed in claim 1, wherein a voice message or text message identifying the anatomical feature is also output when the trigger condition is fulfilled.

10. The computer-implemented method as claimed in claim 1, wherein a cluster analysis is performed to evaluate the trigger condition, to detect clusters of a geometric factor map describing the local geometric factors, within which all geometric factors achieve or exceed a specified threshold, wherein the clusters whose dimensions fulfill a specified selection condition are selected as the respective area to which an anatomical feature is assigned.

11. The computer-implemented method as claimed in claim 1, wherein a detection algorithm embodied by a model trained using machine learning, detects anatomical features in the image data set or in image data that is reconstructed depending on the image data set, wherein selection of the detection algorithm depends on the acquired body region, and/or wherein the acquired body region is processed as part of input data of the detection algorithm, wherein the area is assigned to one of the detected anatomical features depending on a relative position of the respective area in relation to the detected anatomical features.

12. A computer-implemented method for training a model trained using machine learning, which is utilized in the computer-implemented method as claimed in claim 1 for determining the at least one processing parameter of the processing algorithm, comprising:
acquiring multiple training data sets, which each comprise input data and target output data for the trained model, wherein the target output data specifies a respective target value for the respective processing parameter to be determined;
training the trained model based on the input data and the target output data; and
providing the trained model.

13. A processing device programmed to carry out the computer-implemented method as claimed in claim 1.

14. A magnetic resonance device comprising a processing device as claimed in claim 13.

15. A non-transitory computer-readable medium comprising a computer program with instructions, which when executed on a data processing device, carry out the computer-implemented method as claimed in claim 1.
